(19) United States
(12) Patent Application Publication
(10) Pub. No.: US 2025/0268738 A1
(43) Pub. Date: Aug. 28, 2025

(54) BALLOON CATHETER ASSEMBLY

(71) Applicant: Eddy Pump Corporation, El Cajon, CA (US)

(72) Inventors: Phillip RHYNER, El Cajon, CA (US); Kevin KHEDER, El Cajon, CA (US)

(21) Appl. No.: 19/065,100

(22) Filed: Feb. 27, 2025

Related U.S. Application Data
(60) Provisional application No. 63/559,092, filed on Feb. 28, 2024.

(51) Int. Cl. A61F 2/958; A61F 2/95

(52) U.S. Cl. CPC A61F 2/958; A61F 2002/9534; A61F 2250/0096

(57) ABSTRACT

The present disclosure provides a balloon catheter assembly configured to detect a stent and accurately locate a retention balloon within the stent. In an embodiment, a balloon catheter assembly in accordance with the present disclosure includes an elongated catheter tube, a retention balloon and a locating sensor. The retention balloon is disposed on the catheter tube and is configured to be inflated from a deflated state to an inflated state. The locating sensor is configured to detect a position of the retention balloon relative to a stent. In an embodiment, the balloon catheter assembly includes or is connected to a control unit configured to detect the position of the retention balloon relative to the stent and determine when the retention balloon is appropriately positioned to widen the stent.

### PRIORITY

This application claims priority to U.S. Provisional Application No. 63/559,092, filed Feb. 28, 2024, entitled “Balloon Catheter Assembly,” the entire disclosure of which is incorporated herein by reference.

### BACKGROUND

### Technical Field

The present disclosure generally relates to a balloon catheter assembly. More specifically, the present disclosure relates to a balloon catheter assembly configured to accurately position a retention balloon relative to a stent.

### Background Information

A stent is a small tube typically used to hold open a passage in the body such as a weakened or narrowed body vessel. The balloon catheter stent includes a catheter that is fed to the body vessel in which the stent is to be disposed. A guide wire is fed through the catheter to the body vessel. A stent is disposed on a deflated balloon that is connected to the guide wire to position the stent in the body vessel. When the stent is located in the desired position, the balloon is inflated to widen the vessel and to expand the stent to fit the inner wall of the body vessel. The catheter, guide wire and balloon are then removed, leaving the stent in place to maintain the widened vessel.

X-ray imaging, such as fluoroscopy, is typically used to guide the balloon catheter stent to the target area in the body vessel. The X-ray imaging can pose radiation hazards to the patient and to those involved in the stent placement procedure. The X-ray imaging can also provide poor imaging of the body vessel in where the stent is placed. Stent localization is also challenging with fluoroscopy due to a material of the stent.

### SUMMARY

Applicant has determined that a need exists for a balloon catheter assembly capable of locating a retention balloon relative to a stent. The present disclosure provides systems and methods of locating a retention balloon of a balloon catheter assembly relative to a stent without requiring x-ray imaging.

The disclosed balloon catheter assemblies accurately position and/or reposition a retention balloon within a stent, particularly when locating a previously placed stent within a body vessel. The disclosed balloon catheter assemblies also accurately position of the retention balloon at the opening ends of the stent, reducing stent failure and other issues related to improper stent positioning and/or deployment.

In view of the state of the known technology, one aspect of the present disclosure is to provide a balloon catheter assembly. The balloon catheter assembly includes an elongated catheter tube, a retention balloon and a locating sensor. The retention balloon is disposed on the elongated catheter tube. The retention balloon is inflatable from a deflated state to an inflated state. The locating sensor is configured to detect a position of the retention balloon relative to a stent.

A second aspect of the present disclosure is to provide another balloon catheter assembly. The balloon catheter assembly includes an elongated catheter tube, a retention balloon, a locating sensor and a control unit. The retention balloon is disposed on the elongated catheter tube and is inflatable from a deflated state to an inflated state. The locating sensor is located on the elongated catheter tube proximal to the retention balloon. The control unit is operatively connected to the locating sensor. The control unit is configured to determine a position of the retention balloon with respect to an existing stent in a body vessel using feedback from the locating sensor.

A third aspect of the present disclosure is to provide a method of adjusting a stent within a vessel. The method includes inserting a balloon catheter assembly having a retention balloon and a locating sensor into a body vessel having a stent therein, detecting a position of the retention balloon relative to the stent using feedback from a locating sensor, positioning the retention balloon within the stent using feedback from the locating sensor, inflating the retention balloon while the retention balloon is positioned within the stent, and deflating the retention balloon for removal of the balloon catheter assembly from the body vessel.

The present disclosure provides a balloon catheter assembly that enables an operator to accurately position and/or reposition a retention balloon within a stent, particularly when locating a previously placed stent within a body vessel. Moreover, the present disclosure also provides a balloon catheter assembly that can accurately position of the retention balloon at the opening ends of the stent, reducing stent failure and other issues related to improper stent positioning and/or deployment.

Other objects, features, aspects and advantages of the disclosed balloon catheter assemblies will become apparent to those skilled in the art from the following detailed description, which, taken in conjunction with the annexed drawings, discloses an exemplary embodiment of a balloon catheter assembly.

### BRIEF DESCRIPTION OF THE DRAWINGS

Referring now to the attached drawings which form a part of this original disclosure:

FIG. 1 is a perspective view of a first example embodiment of a balloon catheter assembly in accordance with the present disclosure;

FIG. 2 is a perspective view of the catheter tube forming a part of the balloon catheter assembly of FIG. 1;

FIG. 3 is a perspective view of a second example embodiment of a balloon catheter assembly in accordance with the present disclosure;

FIG. 4 is a perspective view of a third example embodiment of a balloon catheter assembly in accordance with the present disclosure; and

FIG. 5 is an example embodiment of a method of inserting or adjusting a stent using a balloon catheter assembly in accordance with the present disclosure.

Throughout the drawing figures, like reference numerals will be understood to refer to like parts, components and structures.

### DETAILED DESCRIPTION

Selected exemplary embodiments will now be explained with reference to the drawings. It will be apparent to those skilled in the art from this disclosure that the following descriptions of the exemplary embodiments are provided for illustration only and not for the purpose of limiting the invention as defined by the appended claims and their equivalents.

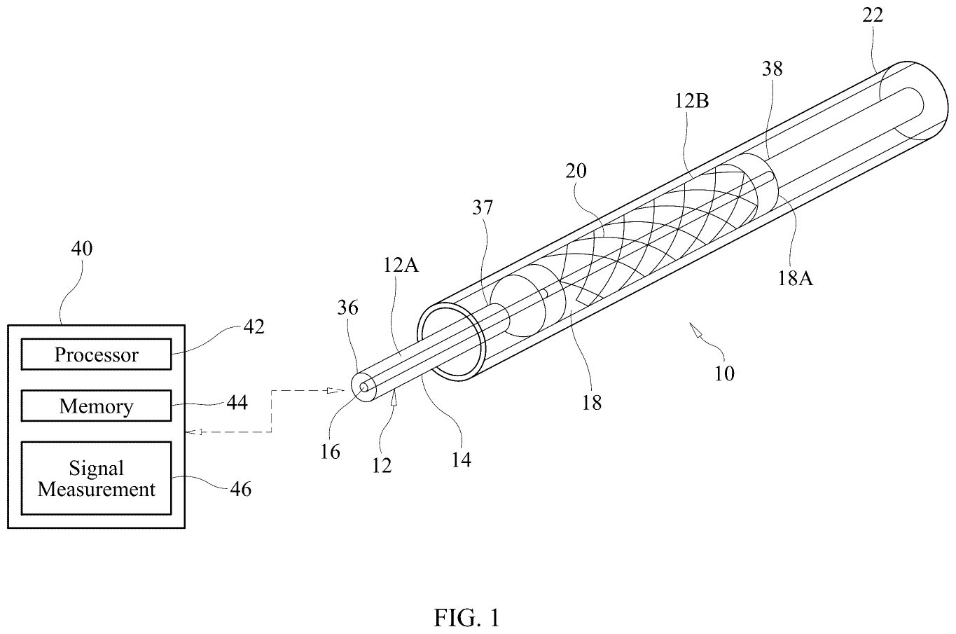

FIG. 1 illustrates a first example embodiment of a balloon catheter assembly 10 in accordance with the present disclosure. In the illustrated embodiment, the balloon catheter assembly 10 generally includes a catheter tube 12 having a shielding surface 14, a locating sensor unit 16 (e.g., an antenna) located within the catheter tube 12, and a retention balloon 18 configured to be inflated to expand a stent 20 to fit the inner wall of a body vessel 22 and/or widen the body vessel 22. As discussed in more detail below, the balloon catheter assembly 10 can generally include a new stent 20 and/or or the balloon catheter assembly 10 can be used in combination with an existing stent 20 already implanted in a body vessel 22. In other words, the balloon catheter assembly 10 can be used to implant a new stent 20 within a body vessel 22, or the balloon catheter assembly 10 can be inserted into a body vessel 22 that has already been widened with an existing stent 20 and can be used to further adjust or widen the existing stent 20.

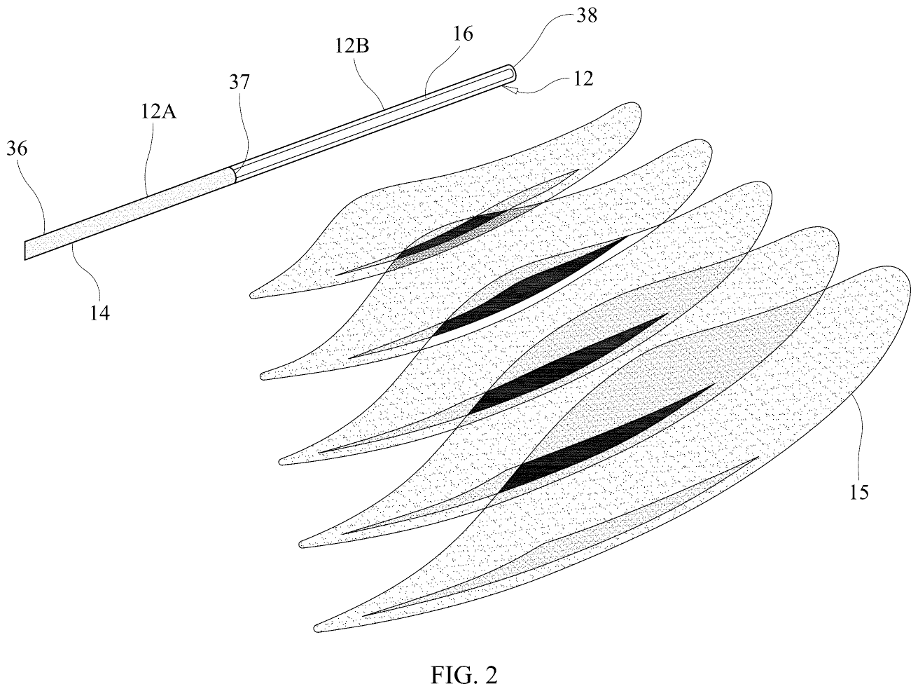

FIG. 2 illustrates an example embodiment of the catheter tube 12. The catheter tube 12 is an elongated hollow tube with a hollow inner space for locating the locating sensor 16 and/or enabling inflation or deflation of the retention balloon 18. In the illustrated embodiment, the catheter tube 12 includes a first portion 12A and a second portion 12B. The first portion 12A extends from a first end 36 to an intermediate point 37. The second portion 12B extends from the intermediate point 37 to a second end 38 opposite the first end 36. The length of the second portion 12B of the catheter tube 12 is preferably substantially equal to a length of the retention balloon 18 being adjusted. For example, the second portion 12B can be between approximately 8 mm and 50 mm in length.

In the illustrated embodiment, the first portion 12A of the catheter tube 12 includes a shielding surface 14. The shielding surface 14 can include a foil. More specifically, the first portion 12A is lined with foil around the inner and/or outer surface thereof from the first end 36 to the intermediate point 37. The foil can be, for example, a metal such as aluminum. The foil creates a shield for the locating sensor 16. More specifically, the foil-lined first portion 12A of the catheter tube 12 is conductive to ground as a shield for receiving the locating sensor 16 inside the catheter tube 12. The second portion 12B of the catheter tube 12 is not lined with foil and does not otherwise including a shielding surface. The non-foil wrapped or non-shielding second portion 12B of the catheter tube 12 is preferably substantially equal to the length of the retention balloon 18.

In the illustrated embodiment, the locating sensor 16 includes an antenna configured to generate an electromagnetic induction (EMI) measurement region. The shielding surface 14 ensures that the EMI measurement region is only generated in the area of the retention balloon 18. The foil-lined first portion 12A of the catheter tube 12 attenuates the radio waves 15 emitted by the locating sensor 16, causing the EMI measurement region only at the second portion 12B of the catheter tube 12 corresponding to the retention balloon 18.

In the illustrated embodiment, the locating sensor 16 (e.g., antenna) is inserted in the catheter tube 12 and terminates at a distal end 18A of the retention balloon 18. More specifically, and as seen in FIG. 1, the antenna extends through the first portion 12A of the catheter tube 12, from the first end 37 and past the intermediate point 37 to terminate near the second end 38 of the catheter tube 12 near the distal end 18A of the retention balloon 18. The antenna conducts a signal to the exterior of the patient's body vessel 22. The antenna operatively connects to a control unit 40 including a signal measurement unit 46, which receives feedback from the antenna regarding an intensity and/or a rate of change of energy (dB) detected in the EMI measurement region. For example, a distal end of a wire extending from the antenna can plug into the signal measurement unit 46. The signal measurement unit 46 runs algorithm utilizing the feedback regarding the energy level and/or the rate of change of the energy level in-situ to detect a relative location of the retention balloon 18 within the stent 20.

As the locating sensor 16 (i.e., antenna) enters and exits the stent 20, the conductive nature of the material of the stent 20 reduces the energy level of the received signal to the locating sensor 16. The reduced energy level of the received signal causes recognizable and repeatable radio energy reduction patterns. The signal measurement unit 46 can adjust the frequency of the transmitted radio waves 15 to match a stent 20 wire spacing to tune the effectiveness of the stent attenuation capability. The signal measurement unit 46 can also adjust the radio energy to counteract a depth of detection due to natural attenuation of the tissue. The signal measurement unit 46 can further adjust the radio energy to use minimal electromagnetic radiation required for a depth of tissue that the radio energy requires for propagation, due to attenuation of the tissue due to salinity. The signal measurement unit 46 indicates when the retention balloon 18 is centered in the stent 20 to ensure proper positioning of the retention balloon 18 within the stent 20 in the body vessel 22.

The retention balloon 18 is configured to inflate from a deflated state to an inflated state, and to then deflate from the inflated state back to the deflated state. The retention balloon 18 can be made from a variety of materials including plastics, resins and polymers. In an embodiment, the control unit 40 monitors and/or controls the inflation and deflation of the retention balloon 18, for example, by monitoring pressure within the catheter tube 12 and/or the retention balloon 18. In an embodiment, the control unit 40 can control inflation of the retention balloon to place the retention balloon 18 in a plurality of different inflated states as needed. For example, the control unit 40 can cause the retention balloon 18 to be inflated to a first inflated state, a second inflated state with a larger radius than the first inflated state, a third inflated state with a larger radius that the first and second inflated states, etc. In an embodiment, the retention balloon 18 can be approximately 10 to 20 mm in length.

The stent 20 is configured to be placed into a body vessel 22 to widen the body vessel 22. The catheter tube 12 is then inserted into the body vessel 22 that has been widened with the stent 20. In other words, FIG. 1 illustrates a stent 20 that has been previously placed in a body vessel 22. When the balloon catheter assembly 10 has accurately positioned the deflated retention balloon 18 within the stent 20, the balloon catheter assembly 10 inflates the retention balloon 18 to adjust or widen the stent 20. The balloon catheter assembly 10 including both the catheter tube 12 and the retention balloon 18 are then removed. The stent 20 remains positioned within the body vessel 22 to maintain the widened state of the body vessel 22.

In the illustrated embodiment, the body vessel 22 is a coronary artery. The body vessel 22 can include a lesion caused by arterial stenosis, which narrows the artery and reduces blood flow to organs and limbs. In other embodiments, the body vessel 22 can also include, for example, an airway, an aneurism, a stomach, a bile duct, a ureter, or another vessel in the body that needs to be widened.

In an embodiment, the stent 20 is initially placed in the body vessel 22 in a conventional manner. For example, a body vessel 22 including a lesion is accessed with a guiding catheter. The lesion is wired with guidewire and predilated with a retention balloon. The retention balloon is embedded within a catheter in the deflated state, and can be inflated and deflated multiple times at the site to dilate the lesion. The lesion may be focal or diffuse, such that the lesion can have a range between approximately 5 mm and approximately 50 mm in length. Retention balloons often require multiple inflations and deflations to dilate stenoses having a length longer than that of the retention balloon. The stent 20 is placed to cover the lesion. Stents are typically between approximately 8 mm and 50 mm in length. Preferably, the stent 20 extends approximately 5 mm beyond the ends of the lesion to ensure proper coverage of the vessel blockage.

In the embodiment of FIG. 1, the balloon catheter assembly 10 is used after the stent 20 is placed in the vessel 22 and the initial stent catheter is removed. The balloon catheter assembly 10 slides over the guidewire (e.g., a coronary guidewire) to accurately identify stent material to ensure that the retention balloon 18 is properly positioned within the previously placed stent 20 prior to inflating the retention balloon 18. In the illustrated embodiment, the length of the retention balloon 18 is between approximately 10 and 20 mm in length and the stent 20 has a length between approximately 15 and 40 mm. The retention balloon 18 can require several inflations when the stent 20 has a length greater than that of the retention balloon 18. Prior to each inflation, the position of the retention balloon 18 relative to the stent 20 is determined by the signal measurement unit 46 to ensure proper positioning of the retention balloon 18 relative to the stent 20.

In another embodiment, the stent 20 can be disposed on the retention balloon 18 prior to placement of the stent 20 within the body vessel 22. The catheter tube 12 can be inserted in a vessel 22 to be widened, as shown in FIG. 1. When the deflated retention balloon 18 is accurately positioned, the retention balloon 18 is inflated to position the stent 20 in the vessel 22. The catheter tube 12 and the retention balloon 18 are then removed. The stent 20 remains positioned within the vessel 22 to maintain the widened state of the vessel 22.

In the illustrated embodiment, the balloon catheter assembly 10 includes or is operatively connected to a control unit 40. The control unit 40 preferably includes a microcomputer 42 with a memory 44 and a control program that controls and/or is operatively connected to one or both of the locating sensor 16 and the retention balloon 18. The control unit 40 can also include other conventional components such as an input interface circuit, an output interface circuit, and storage devices such as a ROM (Read Only Memory) device and a RAM (Random Access Memory) device. The memory circuit stores processing results and control programs for operations that are run by the processor circuit. The microcomputer 42 of the controller 40 is programmed to control the locating sensor 16 and/or the retention balloon 18. The control unit 40 can be operatively coupled to the balloon catheter assembly 10 in a conventional manner. The control unit 40 is capable of selectively controlling any of the components of the balloon catheter assembly 10 in accordance with one or more control programs.

In the illustrated embodiment, the control unit 40 (and/or the signal measurement unit 46) causes the antenna of the locating sensor 16 to generate an EMI measurement signal when the antenna is in the vicinity of the stent 20 to be adjusted. The control unit 40 then continuously analyzes the intensity and/or rate of change (dB) of the signal for recognizable and repeatable radio energy reduction patterns. If needed, the control unit 40 also adjusts the frequency of the transmitted radio waves 15 to match a stent 20 wire spacing to tune the effectiveness of the stent attenuation capability. The control unit 40 determines that the retention balloon 18 is accurately positioned within the stent 20 when the signal measurement unit 46 detects the recognizable energy pattern.

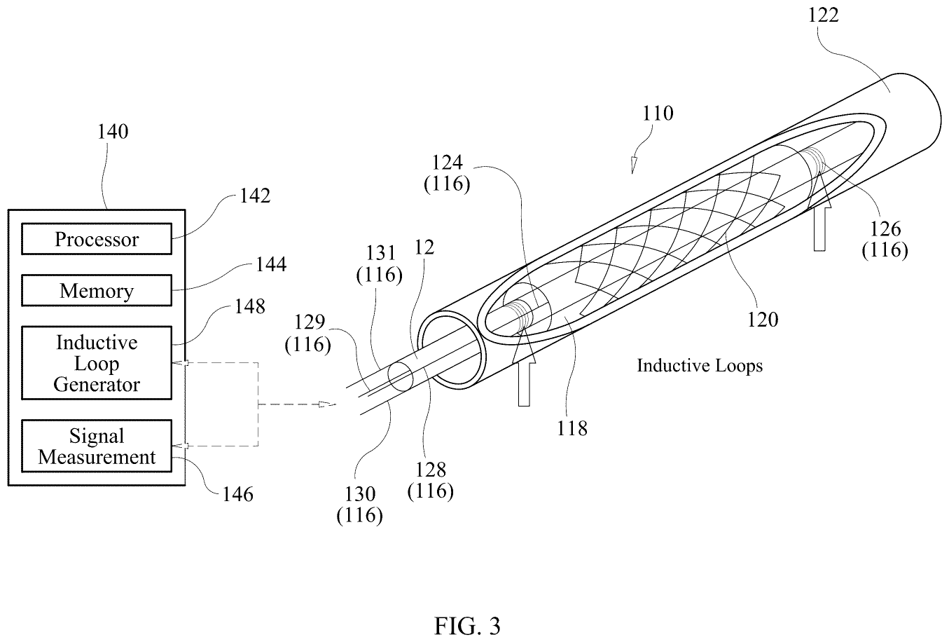

FIG. 3 illustrates a second example embodiment of a balloon catheter assembly 110 in accordance the present disclosure. The balloon catheter stent 110 includes a catheter tube 112, at least one locating sensor 116 and a retention balloon 118. As with the first embodiment, the balloon catheter assembly 10 can be used to implant a new stent 120 within a body vessel 122, or the balloon catheter assembly 110 can be inserted into a body vessel 122 that has already been widened with an existing stent 120 and can be used to further adjust or widen the existing stent 120.

In the illustrated embodiment, the balloon catheter assembly 110 includes a first coil 124 and a second coil 126. More specifically, the locating sensor 116 includes the first coil 124 and the second coil 126. The first coil 124 and the second coil 126 are disposed on the catheter tube 112 at each opposite end of the retention balloon 118. The catheter tube 112 and the retention balloon 118 can be formed as discussed above with respect to the catheter 12 and the retention balloon 18 of the first embodiment of the balloon catheter assembly 10.

In the illustrated embodiment, the balloon catheter assembly 110 includes one or more of a first wire 128, a second wire 129, a third wire 130 and a fourth wire 131. More specifically, the locating sensor 116 includes the first wire 128, the second wire 129, the third wire 130 and the fourth wire 131. The first wire 128 extends from the first coil 124 and is operatively connected to a signal measurement unit 146 of the control unit 140. The second wire 129 extends from the first coil 124 and is operatively connected to an inductive loop generator 148 of the control unit 140. The third wire 130 extends from the second coil 126 and is operatively connected to the signal measurement device 146. The fourth wire 131 extends from the second coil 126 and is operatively connected to the inductive loop generator 148. In other words, each of the first coil 124 and the second coil 126 has a pair of discrete wires. The first coil 124 and the second coil 126 resonate from electricity transmitted to the first coil 124 and the second coil 126 from the inductive loop generator 148.

As with the first example embodiment, the balloon catheter assembly 110 includes or is operatively connected to a control unit 140. The control unit 140 preferably includes a microcomputer 142 with a memory 144 and a control program that controls and/or is operatively connected to one or both of the locating sensor 116 (e.g., the first coil 124 and the second coil 126) and the retention balloon 118. The controller 140 can also include other conventional components such as an input interface circuit, an output interface circuit, and storage devices such as a ROM (Read Only Memory) device and a RAM (Random Access Memory) device. The memory circuit stores processing results and control programs for operation that are run by the processor circuit. The microcomputer of the control unit 140 is programmed to control the locating sensor 116 and/or the retention balloon 118. The control unit 140 can be operatively coupled to the balloon catheter assembly 10 in a conventional manner. The control unit 40 is capable of selectively controlling any of the components of the balloon catheter assembly 10 in accordance with one or more control programs.

In the illustrated embodiment, the control unit 140 includes or controls the signal measurement unit 146 and/or the inductive loop generator 148. The signal measurement unit 146 is configured to measure the resonant frequency and/or amplitude at the first coil 124 and the second coil 126 using the first wire 128 and the third wire 130. The inductive loop generator 148 is configured to generates an inductive loop using the second wire 129 and the fourth wire 131. In an embodiment, the signal measurement unit 146 and the inductive loop generator 148 can be separate units forming the control unit 140.

When a change in a conductive material such as a stent 120 is proximal to either the first coil 124 or the second coil 126, the resonant frequency changes. The signal measurement unit 146 is therefore configured to detect a minimal change of frequency and/or amplitude, which at the original resonant frequency indicates the proximity of either the first coil 124 or the second coil 126 to conductive material such as the stent 120. An equal change in the signal from both the first coil 124 and the second coil 126 indicates that the stent 120 is centered between the first coil 124 and second coils 126, thereby indicating that the stent 120 is centered over the retention balloon 118 since the first coil 124 and the second coil 126 are located at opposite ends of the retention balloon 118.

In an embodiment, the control unit 140 is configured to continuously detect the resonant frequency and/or amplitude at the first coil 124 and the second coil 126 and issue an alert that the retention balloon 118 is properly positioned upon detection of an equal change in signal from both the first coil 124 and the second coil 126. The control unit 40 can also use an unequal change in signal from the first coil 124 compared to the second coil 126 to determine a current position of the stent 120 with respect to the first coil 124 and/or second coil 126. That is, the control unit 140 can also determine that one of the first coil 124 and the second coil 126 is located outside of the stent 120 while the other of the first coil 124 and the second coil 126 is located within the stent 120 based on the unequal frequencies. The control unit 140 can use this determination to instruct a doctor the direction to move the retention balloon 118 with respect to the stent 120 to center the retention balloon 118 within the stent 120. In an embodiment, the control unit 140 can also automatically cause movement of the retention balloon 118 in the appropriate direction. In another embodiment, the control unit 140 automatically inflates the retention balloon 118 upon determining that there is an equal change in signal from both the first coil 124 and the second coil 126. In an embodiment, the radial distance from the first coil 124 and the second coils 126 to the stent 120 can be less than the radius of the first coil 124 and the second coil 126.

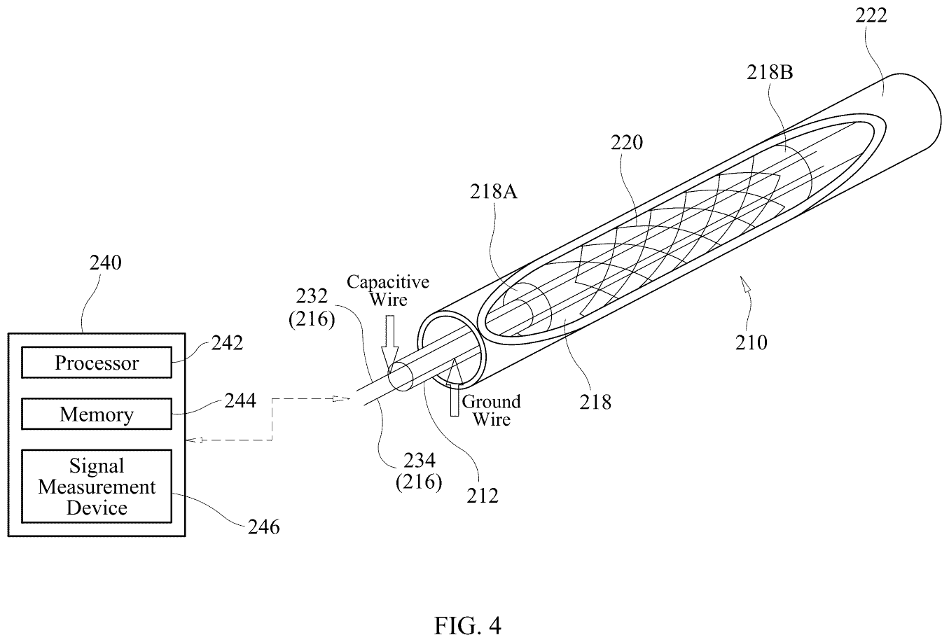

FIG. 4 illustrates another example embodiment of a balloon catheter assembly 210 in accordance with the present disclosure. The balloon catheter assembly 210 includes a catheter tube 212, a locating sensor 216 and a retention balloon 218. As with the first and second embodiments, the balloon catheter assembly 210 can be used to implant a new stent 220 within a body vessel 222, or the balloon catheter assembly 210 can be inserted into a body vessel 222 that has already been widened with an existing stent 220 and can be used to further adjust or widen the existing stent 220.

In the illustrated embodiment, the retention balloon 218 has a first end 218A and a second end 218B disposed on the catheter tube 212. The balloon catheter assembly 210 also includes a conductive wire 232 and a ground wire 234. More specifically, the locating sensor 216 includes the conductive wire 232 and the ground wire 234. The conductive wire 232 is disposed within the catheter tube 212. More specifically, the conductive wire 132 extends through the first end 218A of the retention balloon 218 and past the second end 218B of the retention balloon 218. The ground wire 234 also extends through the first end 218A of the balloon 218 and past the second end 218B of the retention balloon 218. The conductive wire 232 is disposed proximal to the ground wire 234 in the catheter tube 212. The catheter tube 212 and the retention balloon 218 can be formed as discussed above for the catheters 12, 112 and retention balloons 18, 118 of the first and second embodiments of the balloon catheter assemblies 10, 110.

In the illustrated embodiment, the balloon catheter assembly 210 includes or is operatively connected to a control unit 240. The control unit 240 preferably includes a microcomputer 242 with a memory 244 and a control program that controls and/or is operatively connected to one or both of the locating sensor 216 and the retention balloon 218. The controller 240 can also include other conventional components such as an input interface circuit, an output interface circuit, and storage devices such as a ROM (Read Only Memory) device and a RAM (Random Access Memory) device. The memory circuit stores processing results and control programs for operation that are run by the processor circuit. The microcomputer of the control unit 240 is programmed to control the locating sensor 216 and/or the retention balloon 218. The control unit 240 can be operatively coupled to the balloon catheter assembly 210 in a conventional manner. The control unit 240 is capable of selectively controlling any of the components of the balloon catheter assembly 210 in accordance with one or more control programs.

In the illustrated embodiment, the control unit 240 includes a signal measurement unit 246. The signal measurement unit 246 monitors the capacitance of the conductive wire 232. The presence of the stent 220 shorts the capacitive area and decreases the capacitance of the conductive wire 232, so the signal measurement unit 246 is configured to detect the decreased capacitance of the conductive wire to determine where the retention balloon 218 is located with respect to the stent 220 and to determine when the retention balloon 218 is accurately positioned with respect to the stent 220.

In the illustrated embodiment, the signal measurement unit 242 charges the conductive wire 232 to a low voltage. The conductive wire 232 is then isolated and the time change in which the voltage decreases a prescribed amount is directly related to the capacitance of the insulated area around the conductive wire 232. The capacitance decreases dramatically with conductors shorting the area between the conductive wire, or capacitive probe, 232 and the ground wire 234. The signal measurement unit 242 is configured to detect the change in capacitance and issue an alert that the retention balloon 218 is properly positioned based on the decrease in capacitance. In an embodiment, the control unit 240 automatically inflates the retention balloon 218 upon detection of the change in capacitance.

In an embodiment, the conductive wire 232 is run in the catheter tube 212 to use the air as an insulator to keep the change of capacitance to only the area of the catheter tube 212 on which the retention balloon 218 is disposed. The position of the ground wire 234 relative to the conductive wire 232 can improve performance of the balloon catheter stent 210.

In an embodiment, the locating device 16, 116, 216 can further includes a sensor configured to detect a material of the stent 20, 120, 220 to facilitate locating a position of a retention balloon 218 relative to the stent 20, 120, 220.

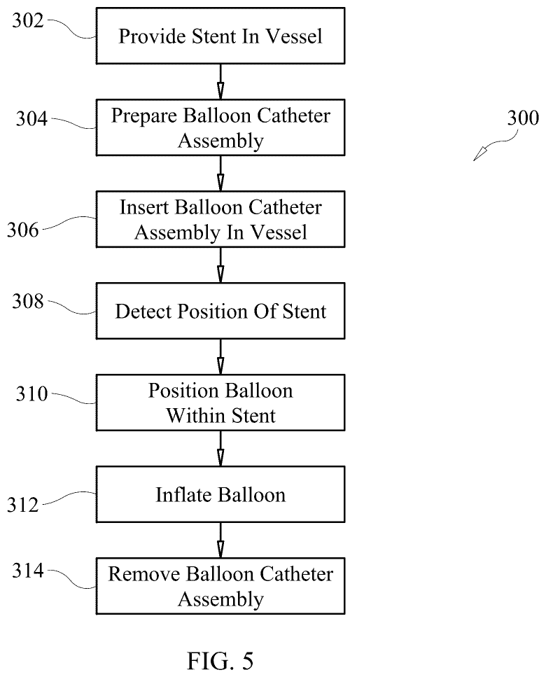

FIG. 5 illustrates an example embodiment of a method 300 of using a balloon catheter assembly 10, 110, 210 in accordance with the present disclosure. One or more of the steps of the method 300 can be performed in accordance with control programs stored and executed by the control unit 40, 140, 240. Those of ordinary skill in the art will further recognize from this disclosure that certain steps of the method 300 can be changed or omitted without departing from the spirit and scope of the present disclosure.

At step 302 of the method 300, a stent 20, 120, 220 is provided in a patient's vessel. In an embodiment, the stent 20, 120, 220 has been previously inserted in the patient's vessel by a balloon catheter assembly 10, 110, 210 in accordance with the present disclosure. Alternatively, the stent 20, 120, 220 has been previously inserted in the body vessel 20, 120, 220 by a conventional or other type of balloon catheter.

At step 304 of the method 300, a balloon catheter assembly 10, 110, 210 in accordance with the present disclosure is prepared. For example, the balloon catheter assembly 10, 110, 210 can be sterilized and/or operatively connected to control unit 40, 140, 240 as described herein.

At step 306 of the method 300, the balloon catheter assembly 10, 110, 210 is inserted into the patient's body vessel 22, 122, 222. For example, the balloon catheter assembly 10, 110, 210 can be inserted into a patient by making a small incision in a blood vessel and then guiding the portion including the retention balloon 18, 118, 218 toward a general target area where the stent 20, 120, 220 is known to be located. At this point, the retention balloon 18, 118, 218 is in its deflated state.

At step 308 of the method 300, the balloon catheter assembly 10, 110, 210 detects the position of the stent 20, 120, 220. More specifically, the control unit 40, 140, 240 detects the position of the retention balloon 18, 118, 218 relative to the stent 20, 120, 220. In an embodiment, the control unit 40, 140, 240 uses the intensity and/or a rate of change of a radio signal to detect the relative positioning, as discussed above. In an embodiment, the control unit 40, 140, 240 uses one or more resonant frequency changes to detect the relative positioning, as discussed above. In an embodiment, the control unit 40, 140, 240 uses a resonant frequency change indicative of a change in conductive material to detect the relative positioning, as discussed above. In an embodiment, the control unit 40, 140 240 instructs a doctor which direction to move the balloon catheter assembly 10, 110, 210 to center the retention balloon 18, 118, 218 within the stent 20, 120, 220. In an embodiment, the control unit 40, 140 240 uses the detected position to automatically move the balloon catheter assembly 10, 110, 210 to center the retention balloon 18, 118, 218 within the stent 20, 120, 220.

At step 310 of the method 300, control unit 40, 140, 240 determines that the retention balloon 18, 118, 218 is accurately positioned within the stent 20, 120, 220 and ready for inflation. Preferably, the balloon catheter assembly 10, 110, 210 is positioned so that opposite ends of the retention balloon 18, 118, 218 are located on opposite sides of the stent 20, 120, 220. The retention balloon 18, 118, 218 can also be positioned off-center with respect to the stent 20, 120, 220, for example, if the retention balloon 18, 118, 218 will require multiple inflations and deflations due to the stent 20, 120, 220 having a length longer than that of the retention balloon 18, 118, 218. In an embodiment, the control unit 40, 140, 240 instructs the doctor to move the balloon catheter assembly 10, 110, 210 in a particular direction and/or by a particular amount based on the relative positioning determined at step 308. In an embodiment, the control unit 40, 140, 240 is configured to automatically cause the movement of the stent retention balloon 18, 118, 218 in a particular direction and/or by a particular amount based on the relative positioning determined at step 308.

In an embodiment, the control unit determines the location using feedback regarding radio energy attenuation from stent 20 induced EMI. More specifically, the control unit 40 causes the locating sensor 16 (e.g., antenna) to generate an EMI measurement signal and receives feedback regarding the signal. The control unit 40 continuously analyzes the intensity and/or rate of change of the signal for recognizable and repeatable radio energy reduction patterns. The control unit 40 is also configured to adjust the frequency of the transmitted radio waves 15 to match a stent 20 wire spacing to tune the effectiveness of the stent attenuation capability. The control unit 40 determines the retention balloon 18 to be accurately positioned within the stent 20 when the recognizable and repeatable radio energy reduction pattern is detected.

In an embodiment, the control unit 140 determines the location using a loop detector. More specifically, the control unit 140 attempts to detect a resonant frequency change indicative of a change in conductive material. More specifically, the control unit 140 attempts to detect a frequency and/or amplitude change that indicates the proximity of either the first coil 124 or the second coil 126 to a stent 120 as discussed above. The control unit 140 determines the retention balloon 118 to be accurately positioned when there is an equal change in signal from both the first coil 124 and the second coil 126. Alternatively, the control unit 140 can determined the retention balloon 118 to be accurately positioned when there is an unequal change from the first coil 124 and the second coil 126, for example, if the retention balloon 118 will require multiple inflations and deflations due to the stent 120 having a length longer than that of the retention balloon 118.

In an embodiment, the control unit determines the location based on a decrease in capacitance at the locating sensor 216. More specifically, the control unit 240 monitors the capacitance of a conductive wire 232 located proximal to the retention balloon 218. The control unit 240 is configured to detect a change in capacitance indicating that the retention balloon 218 to be accurately positioned within a stent 220.

At step 312, the retention balloon 18, 118, 218 is inflated into its inflated state. In an embodiment, the control unit 40, 140, 240 automatically causes the retention balloon 18, 118, 218 to be inflated upon determination that the retention balloon 18, 118, 218 is accurately positioned within the stent 20, 120, 220. In an embodiment, the control unit 40, 140, 240 further determines an amount of inflation needed based on the desired widening of the stent 20, 120, 220. In an embodiment, the control unit 40, 140, 240 inflates the retention balloon 18, 118, 218 multiple times in multiple locations.

At step 314, the retention balloon 18, 118, 218 is deflated back to its deflated state and the balloon catheter assembly 10, 110, 210 is removed from the body vessel 22, 122, 222. In an embodiment, the control unit 40, 140, 240 automatically deflates the retention balloon 18, 118, 218 after determining that the retention balloon 18, 118, 218 has been sufficiently inflated to adjust the stent 20, 120, 220 as needed for the patient.

Those of ordinary skill in the art will recognize from this disclosure that the elements of any of the balloon catheter assemblies 10, 110, 210 disclosed herein can be combined with elements of any of the other balloon catheter assembly 10, 110, 210 disclosed herein.

The foregoing detailed description of the certain exemplary embodiments has been provided for the purpose of explaining the principles of the invention and its practical application, thereby enabling others skilled in the art to understand the invention for various exemplary embodiments and with various modifications as are suited to the particular use contemplated. This description is not necessarily intended to be exhaustive or to limit the invention to the exemplary embodiments disclosed. Any of the exemplary embodiments and/or elements disclosed herein may be combined with one another to form various additional embodiments not specifically disclosed. Accordingly, additional embodiments are possible and are intended to be encompassed within this specification and the scope of the appended claims. The specification describes specific examples to accomplish a more general goal that may be accomplished in another way.

### General Interpretation of Terms

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” or “element” when used in the singular can have the dual meaning of a single part or a plurality of parts unless otherwise stated.

Also, it will be understood that although the terms “first” and “second” may be used herein to describe various components, these components should not be limited by these terms. These terms are only used to distinguish one component from another. Thus, for example, a first component discussed above could be termed a second component and vice versa without departing from the teachings of the present invention. The term “attached” or “attaching”, as used herein, encompasses configurations in which an element is directly secured to another element by affixing the element directly to the other element; configurations in which the element is indirectly secured to the other element by affixing the element to the intermediate member(s) which in turn are affixed to the other element; and configurations in which one element is integral with another element, i.e. one element is essentially part of the other element. This definition also applies to words of similar meaning, for example, “joined”, “connected”, “coupled”, “mounted”, “bonded”, “fixed” and their derivatives. Finally, terms of degree such as “substantially”, “about” and “approximately” as used herein mean an amount of deviation of the modified term such that the end result is not significantly changed.

The term “configured” as used herein to describe a component, section or part of a device or element includes hardware and/or software that is constructed and/or programmed to carry out the desired function.

The terms of degree such as “generally”, “substantially”, “about” and “approximately” as used herein mean a reasonable amount of deviation of the modified term such that the end result is not significantly changed.

While only selected embodiments have been chosen to illustrate the present invention, it will be apparent to those skilled in the art from this disclosure that various changes and modifications can be made herein without departing from the scope of the invention as defined in the appended claims. For example, unless specifically stated otherwise, the size, shape, location or orientation of the various components can be changed as needed and/or desired so long as the changes do not substantially affect their intended function. Unless specifically stated otherwise, components that are shown directly connected or contacting each other can have intermediate structures disposed between them so long as the changes do not substantially affect their intended function. The functions of one element can be performed by two, and vice versa unless specifically stated otherwise. The structures and functions of one embodiment can be adopted in another embodiment. It is not necessary for all advantages to be present in a particular embodiment at the same time. Every feature which is unique from the prior art, alone or in combination with other features, also should be considered a separate description of further inventions by the applicant, including the structural and/or functional concepts embodied by such feature(s). Thus, the foregoing descriptions of the exemplary embodiments according to the present invention are provided for illustration only, and not for the purpose of limiting the invention as defined by the appended claims and their equivalents.

What is claimed is:

1. A balloon catheter assembly, comprising:
an elongated catheter tube;
a retention balloon disposed on the elongated catheter tube, the retention balloon being inflatable from a deflated state to an inflated state; and
a locating sensor configured to detect a position of the retention balloon relative to a stent.

2. The balloon catheter assembly of claim 1, wherein
the position of the retention balloon relative to the stent is determined prior to inflating the balloon to the inflated state.

3. The balloon catheter assembly of claim 1, wherein
the locating sensor includes a foil surface around a portion of the catheter tube and an antenna disposed in the catheter tube and extending through the balloon.

4. The balloon catheter assembly of claim 3, wherein
the antenna terminates at an end of the retention balloon.

5. The balloon catheter assembly of claim 1, wherein
the locating sensor includes a first conductive coil disposed at a first location on the catheter tube and a second conductive coil disposed at a second location on the catheter tube, the first location and second location being located at opposite ends of the retention balloon.

6. The balloon catheter assembly of claim 1, wherein
the locating sensor includes a conductive wire disposed in the catheter tube and extending through the retention balloon and a ground wire disposed in the catheter tube and extending through the retention balloon.

7. The balloon catheter assembly of claim 1, wherein
the stent is disposed in an artery.

8. The balloon catheter assembly of claim 1, wherein
the stent is disposed on the retention balloon in the deflated state.

9. A balloon catheter assembly, comprising
an elongated catheter tube;
a retention balloon disposed on the elongated catheter tube, the retention balloon being inflatable from a deflated state to an inflated state;
a locating sensor located on the elongated catheter tube proximal to the retention balloon; and
a control unit operatively connected to the locating sensor, the control unit configured to determine a position of the retention balloon with respect to an existing stent in a body vessel using feedback from the locating sensor.

10. The balloon catheter assembly of claim 9, wherein
the locating sensor includes a first coil and a second coil,
the first coil is located on the elongated catheter tube at one end of the retention balloon, and
the second coil is located on the elongated catheter tube at an opposite end of the retention balloon.

11. The balloon catheter assembly of claim 9, wherein
the locating sensor includes a conductive wire disposed in the catheter tube and extending through the retention balloon and a ground wire disposed in the catheter tube and extending through the retention balloon.

12. The balloon catheter assembly of claim 9, wherein
the control unit is configured to use feedback regarding an intensity or a rate of change of a radio signal to determine a relative positioning of the locating sensor and the stent.

13. The balloon catheter assembly of claim 9, wherein
the control unit is configured to use feedback regarding one or more resonant frequency changes to determine a relative positioning of the locating sensor and the stent.

14. The balloon catheter assembly of claim 9, wherein
the control unit is configured to use feedback regarding a resonant frequency change indicative of a change in conductive material to determine a relative positioning of the locating sensor and the stent.

15. A method of adjusting a stent within a vessel, the method comprising inserting a balloon catheter assembly having a retention balloon and a locating sensor into a vessel having a stent therein;
detecting a position of the retention balloon relative to the stent using feedback from a locating sensor;
positioning the retention balloon within the stent using feedback from the locating sensor;
inflating the retention balloon while the retention balloon is positioned within the stent; and
deflating the retention balloon for removal of the balloon catheter assembly from the vessel.

16. The balloon catheter assembly of claim 15, wherein
detecting the position of the stent relative to the balloon catheter assembly includes detecting an intensity or a rate of change of a radio signal.

17. The balloon catheter assembly of claim 15, wherein
detecting the position of the stent relative to the balloon catheter assembly includes detecting one or more resonant frequency changes.

18. The balloon catheter assembly of claim 15, wherein
detecting the position of the stent relative to the balloon catheter assembly includes detecting a resonant frequency change indicative of a change in conductive material.

19. The balloon catheter assembly of claim 15, comprising
automatically adjusting the positioning of the retention balloon based on feedback from the locating sensor.

20. The balloon catheter assembly of claim 15, comprising
automatically inflating the retention balloon within the stent upon detecting equal change in signal from both a first coil on one side of the retention balloon and a second coil on an opposite side of the retention balloon.
